(19) United States
(12) Patent Application Publication
(10) Pub. No.: US 2021/0332174 A1
(43) Pub. Date: Oct. 28, 2021

(54) HYDROGEL COMPOSITIONS

(71) Applicants: OPHTALMIC COMPAGNIE, VILLEPINTE (FR); Université de Haute-Alsace, MOLHOUSE (FR)

(72) Inventors: Jean-François STUMBÉ, STRASBOURG (FR); Romain JAGU, DREFFÉAC (FR); Jean-François RUMIGNY, SURZUR (FR); Sophie BISTAC, GALFINGUE (FR); Fanny COUMES, LEUC (FR)

(21) Appl. No.: 17/282,209

(22) PCT Filed: Sep. 30, 2019
(86) PCT No.: PCT/FR2019/052303

§ 371 Date: Apr. 1, 2021

(30) Foreign Application Priority Data
Oct. 2, 2018 (FR) 1859125

(51) Int. Cl. C08F 283/08; C08G 65/332; C08G 65/333; G02B 1/04; G02C 7/04

(52) U.S. Cl. CPC C08F 283/08; C08G 65/3328; C08G 65/33351; A61L 2430/16; G02C 7/04; C08F 2810/20; G02B 1/043

(57) ABSTRACT

A crosslinkable composition comprising: A) at least one polymerisable monomer or a mixture of polymerisable monomers or a mixture of polymerisable monomers and macromonomers bearing at least one polymerisable C═C function of the acrylate, methacrylate, maleate, fumarate, itaconate, citraconate, styrene or vinyl type; B) at least one polyol chosen from oligoglycerols, linear, branched and hyperbranched polyglycerols, or a mixture of hyperbranched polyglycerol and a polyol chosen from oligoglycerols, and linear, branched and hyperbranched polyglycerols, comprising, on average, more than 1 hydroxyl group functionalised with a group bearing a polymerisable C═C function of the acrylate, methacrylate, maleate, fumarate, itaconate, citraconate, styrene or vinyl type; C) a free radical initiator or a mixture of free radical initiators capable of initiating polymerisation by thermal, redox or photochemical means; and D) optionally, at least one additive.

### TECHNICAL FIELD

The present invention relates to crosslinkable and crosslinked compositions, as well as to a hydrogel obtainable starting from the crosslinked composition.

The present invention finds industrial applications in the field of biocompatible materials, and in particular for contact lenses.

In the following description, the references in square brackets ([ ]) refer to the list of references given at the end of the text.

### PRIOR ART

A contact lens, worn on the eye, serves to correct defects of vision such as myopia, astigmatism, or hypermetropia. However, the human eye needs to maintain a certain level of hydration and circulation of oxygen. Thus, the lens in contact with the eye must meet a specification including but not limited to good permeability to oxygen, good comfort, good water retention, or a hydrophilic character.

Contact lenses can be classified in two categories: hard contact lenses, and soft contact lenses, such as hydrogel lenses or lenses of silicone hydrogel.

A hydrogel is a hydrated crosslinked polymer system that contains water in a state of equilibrium. It is typically permeable to oxygen and biocompatible, which makes it a preferred material for producing biomedical devices and in particular contact lenses or intraocular lenses. Soft lenses of hydrogel are generally manufactured from a limited number of base monomers. Which are chosen will depend on the final character that the lens manufacturer wishes to promote:
better permeability to oxygen increasing with the water content or with the silicone content,
better resistance to lipid deposits and to protein deposits in particular,
better resistance to dehydration, which tends to increase with the water content.

In general, in the production of polymer contact lenses, a polymerizable composition of lens precursors is polymerized to form a contact lens product, which is then treated to form a hydrated contact lens. For example, the lens may be prepared by a molding technique, which consists of putting the polymerizable precursor composition in a mold of the desired shape, where it can be polymerized to form a contact lens. Polymerization can be carried out by exposing the polymerizable resin consisting of polymerizable precursors to ultraviolet light or to heat. After polymerization of the composition of lens precursors, the mold parts are separated, and the polymerized contact lens can be removed from the mold, i.e. lifted out or extracted from the mold part.

Conventional hydrogel contact lenses are often the polymerized product of a composition of lens precursors containing hydrophilic monomers such as 2-hydroxyethyl methacrylate (HEMA), methacrylic acid (MAA), and N-vinylpyrrolidone (NVP), which may in addition contain unreactive additives for improving certain properties such as water retention and mechanical properties, such as polyvinyl alcohol or poly(vinyl alcohol) (PVA), polyvinylpyrrolidone (PVP) and polyethylene glycol (PEG), and combinations thereof. The precursor compositions also often contain one or more catalysts and crosslinking agents.

More recently, a new generation of polymers has been developed for further increasing permeability to oxygen. These materials are based on the copolymerization of (meth)acrylate or di(meth)acrylate monomers functionalized with silicone groups with hydrophilic comonomers such as HEMA. The lenses produced starting from these materials were initially designed for prolonged usage, to be worn continuously, 24 hours a day for 15 to 30 days. Although having succeeded in increasing permeability to oxygen, these new materials still have limitations such as adhesion or lipid and protein deposits and dryness, which reduces eye comfort.

There is therefore a real need for new materials that overcome the shortcomings, drawbacks and obstacles of the prior art, in particular having particularly beneficial properties in terms of permeability to oxygen, water content, water loss, Young's modulus, permeability to ions, tensile strength, elongation at break, coefficient of friction and surface roughness.

### DESCRIPTION OF THE INVENTION

After extensive research, the applicant has succeeded in developing a new material that meets the aforementioned needs, and so is particularly suitable for use for making contact lenses.

Surprisingly, the applicant has succeeded in creating a transparent hydrogel that is capable of swelling in an aqueous medium, has good resistance to drying out, and shows very advantageous properties relative to the existing materials, in particular using novel functionalized polymers consisting of oligoglycerols or polyglycerols, in particular linear, branched or hyperbranched polyglycerols or mixtures of hyperbranched polyglycerol with oligoglycerols or with linear polyglycerols, as the main polymer constituent or as a secondary constituent.

These new polymers offer the advantage, because they are highly functionalized, that they have a plurality of properties that allow them for example to be used as a crosslinking agent, and/or as a hydrophilic species, and they are highly permeable to oxygen in the hydrated state.

Numerous combinations are conceivable in order to use the functionalized polymer with a very large number of molecules for obtaining a hydrogel material consisting of a very wide choice of functions acting on the physicochemical and biological properties of the hydrogel.

Advantageously, the medical device is biocompatible, and in particular is suitable for contact with the eyes, and is hydrophilic. It also has particularly interesting properties in terms of permeability to oxygen, water content, water loss, Young's modulus, permeability to ions, tensile strength, elongation at break, coefficient of friction and surface roughness.

Thus, the invention relates firstly to a crosslinkable composition comprising, or consisting of:

A) at least one polymerizable monomer or a mixture of polymerizable monomers or a mixture of monomers and polymerizable macromonomers bearing at least one polymerizable C═C function of the acrylate, methacrylate, maleate, fumarate, itaconate, citraconate, styrenic or vinylic type;

B) at least one polyol selected from oligoglycerols, linear, branched and hyperbranched polyglycerols, a mixture of hyperbranched polyglycerol and of a polyol selected from oligoglycerols and linear, branched and hyperbranched polyglycerols, comprising on average more than 1 hydroxyl group functionalized with a group bearing a polymerizable C═C function of the acrylate, methacrylate, maleate, fumarate, itaconate, citraconate, styrenic or vinylic type;

C) a radical initiator or a mixture of radical initiators that are able to initiate polymerization by thermal, redox or photochemical routes; and

D) optionally at least one additive selected from antioxidants, agents for adjusting physicochemical properties such as Young's modulus, elongation at break and breaking stress, agents for promoting, modulating and/or controlling permeability to oxygen, plasticizers, moistening agents, for example such as PVP, PVA and PEG, lubricants, for example such as hyaluronic acid, viscosity modifiers, compatibilizers, colorants, filtering agents, antimicrobial agents, therapeutic agents and agents against bacterial biofilms.

Regarding constituent A), “polymerizable monomer” means, in the sense of the present invention, any monomer that may contain at least one polymerizable reactive terminal function. It may be a single type of monomer, or a mixture of different monomers, for example 2 different types of monomers, or 3 different types of monomers, or 4 different types of monomers, or more than 4 different types of monomers.

“Polymerizable macromonomers” means, in the sense of the present invention, any oligomers having a polymerizable reactive terminal function, also known by the term monotelechelic macromonomers, or two polymerizable reactive terminal functions, also known by the term α,ω-ditelechelic oligomer, or more than two polymerizable functions, generally three or four.

In the case of a mixture of monomers and macromonomers, it may be a mixture comprising at least one type of monomer and at least one type of macromonomer. There may be, for example, within the mixture, 1 or 2 or 3 or 4 or more than 4 types of monomers, and 1 or 2 or 3 or 4 or more than 4 types of macromonomers. It may for example be a mixture comprising the monomer HEMA (A), reacting with functionalized hyperbranched polyglycerol (B).

The ratio of monomer to macromonomer may be adapted by a person skilled in the art in relation to the desired properties of the hydrogel material to be prepared.

In constituent A), the ethylenically unsaturated group representing the polymerizable C═C function may be selected from the acrylate, methacrylate, maleate, fumarate, itaconate, citraconate, acrylamide or vinyl functions.

Constituent A) may in particular be heterobifunctional. In this case, it may be for example a compound having 1 acrylate group and a methacrylate group, or any other combination of the aforementioned groups.

In constituent A), the monomer or macromonomer may contain one or more, for example 2 or more than 2, fluorinated, silane and/or siloxane groups.

The monomer or macromonomer may be selected for example from:
acrylic acid or methacrylic acid as well as alkyl (meth)acrylates and derivatives thereof, such as 2-hydroxyethylmethacrylate (HEMA), 2-hydroxyethylacrylate (HEA), methylmethacrylate (MMA), methacrylamide, N,N-dimethyl-diacetoneacrylamide, 2-phosphatoethylmethacrylate, mono-, di-, tri-, tetra-, penta-polyethylene glycol acrylates or methacrylates, N-(3-methacrylamidopropyl)-N,N-dimethylamine, N-(3-methacrylamidopropyl)-N,N,N-trimethylamine, N-(3-acrylamido-3-methylbutyl)-N,N-dimethylamine, N-methacrylamide, 3-hydroxypropyl methacrylate, propyl methacrylate, propyl acrylate, butyl methacrylate, butyl acrylate, pentyl acrylate, pentyl methacrylate, N-(1,1-dimethyl-3-oxobutyl)acrylamide, 2-ethyl-2-(hydroxy-methyl)-1,3-propanediol trimethacrylate, butyl(meth)acrylate, 2-hydroxybutyl methacrylate, 3-hydroxy-2-naphthyl methacrylate, N-(formylmethyl)acrylamide, 2-ethoxyethyl methacrylate, 4-t-butyl-2-hydroxycyclohexyl methacrylate, or the isocyanatoalkyl(meth)acrylates such as 2-isocyanatoethylmethacrylate, 2-isocyanatoethyl acrylate, which are commercially available, or can also be synthesized according to the methodology described in document US20010005738 ([1]), or else a compound selected from 4-[(a)-phenyl diazenyl]phenyl-2-methacrylate (4-[(E)-phenyldiazenyl]phenyl-2-methacrylate) or 4-[(a)-phenyl diazenyl]phenyl-2-methacrylate (4-[(E)-phenyldiazenyl]phenyl-2-methacrylate), which are described in document WO2017150786 ([2]), and these last-mentioned compounds may be present at up to about 5 mol % of the acrylates or of the mixture of acrylates,
maleate and fumarate monomers, such as maleic acid, maleic anhydride, dimethyl maleate, diethyl maleate, or higher maleate esters in which the alkyl chain contains 3 to 20 carbon atoms as well as fumaric acid, dimethyl fumarate, diethyl fumarate or the higher fumarate esters in which the alkyl chain contains 3 to 20 carbon atoms,
itaconate monomers; such as itaconic acid, itaconic anhydride, dimethyl itaconate, or higher itaconate esters in which the alkyl chain contains 3 to 20 carbon atoms,
citraconate monomers; such as citraconic acid, citraconic anhydride, dimethyl citraconate, or higher citraconate esters in which the alkyl chain is an alkyl group containing 3 to 20 carbon atoms,
(meth)acrylic monomers that may contain silicone or silane functions; for example tris(trimethylsiloxy)silylpropyl methacrylate, bis(trimethylsiloxy)methylsilylpropyl methacrylate, pentamethyldisiloxanepropyl methacrylate, tris(trimethylsiloxy) silylpropyloxyethyl methacrylate, tris(trimethylsiloxy)-silylpropyl methylacryloxyethylcarbamate, tris(trimethylsiloxy)silylpropylglycerol methacrylate, phenyltetramethyl-disiloxanylethyl acrylate, 3-methacryloxypropylbis(trimethylsiloxy)methylsilane, methyl-di(trimethylsiloxy)methacryloxymethyl silane, (3-methacryloxy-2-hydroxypropyloxy) propyl bis(trimethylsiloxy)methylsilane, 3-acryloxypropyl tris(trimethylsiloxy)silane, 3-acryloxypropyltrichlorosilane, 3-methacryloxypropyl dimethylethoxysilane, 3-methacryloxypropylmethyl diethoxysilane, methacryloxypropylbis(trimethylsiloxy)methylsilane, tristrimethylsilyloxysilylpropyl methacrylate, bis(methacryloxypropyl) tetramethyldisiloxane, acryloxymethyltrimethylsilane, p-(t-butyldimethylsiloxy)styrene, 1,3-bis(3-methacryloxypropyl)tetrakis(trimethylsiloxy)disiloxane, 3-trimethylsilylpropargylmethacrylate, 3-(trimethoxysilyl)propyl acrylate,
vinyl monomers, which may contain silicone or silane functions; for example vinyl(chloromethyl)dimethoxysilane, vinylethoxysiloxane-propylethoxysiloxane, vinyltris(1-methoxy-2-propoxy)silane, vinyltris(2-methoxyethoxy)silane, vinyltrimethoxysilane, vinyltriisopropoxysilane, vinyltriethoxysilane, vinyltrichlorosilane, vinyl tri-t-butoxysilane, o-(vinyloxybutyl)-n-triethoxysilylpropyl carbamate, vinylmethyldimethoxysilane, vinylmethyldiethoxysilane, vinyldimethylethoxysilane, 3-[tris(trimethylsiloxy)silyl] propylvinyl carbamate, triethoxysilylpropyl maleic acid or 1,3,5-trivinyl-1,1,3,5,5-pentamethyltrisiloxane,
fluorinated acrylic and methacrylic monomers, such as dihydroperfluorooctyl acrylate, 1,1-dihydroperfluorobutyl acrylate, the trihydroperfluoroalkyl acrylates, the tetrahydroperfluoroalkylacrylates, tris(trimethylsilyloxy)propyl methacrylate, perfluoro hexylethylthiocarbonylaminoethyl methacrylate, trifluoroethyl methacrylate, hexafluoroisopropyl methacrylate, hexafluorobutyl methacrylate, trifluoroethyl methacrylate,
organosilane prepolymer compounds, such as α,ω-bismethacryloxy-propyl polydimethylsiloxane, polydimethylsiloxanes with vinylic termination(s), diphenylsiloxane-dimethylsiloxane copolymers with a vinylic termination, trifluoropropylmethylsiloxane copolymers with vinylic termination(s), diethylsiloxane-dimethylsiloxane copolymers with vinylic termination(s), ethylsiloxane-dimethylsiloxane copolymers with vinylic termination(s), ethylene-siloxane copolymers with vinylic termination(s), tris(polydimethylsiloxy)silylpropyl methacrylate, polysiloxanylalkyl (meth)acrylates, methacryloxypropyl with polydimethylsiloxane termination, 1,3-divinyl-1,1,3,3-tetramethyldisiloxane, 2-(divinylmethylsilyl)ethyltriethoxysilane, or 1,3-divinyl-1,3-diphenyl-1,3-dimethyldisilane,
fluorinated/silane or chlorinated/silane mixed compounds, such as fluoro-methacryloxypropyl tris(trimethylsiloxy) silane, 3-acryloxy propyl methyl dichlorosilane, 3-acryloxypropyltrichlorosilane, 3-methacryloxypropyl trichlorosilane,
silane/isocyanate compounds such as 3-isocyanatopropyltriethoxysilane, and
vinyl monomers, such as N-vinyl lactams; for example N-vinyl pyrrolidone (NVP)), N-vinyl-N-methyl acetamide (VMA), N-vinyl-N-ethyl acetamide, N-vinyl-N-ethyl formamide, N-vinyl formamide, N-2-hydroxyethyl vinyl carbamate, N-carboxy-alanine N-vinyl ester, preferably CH₃C(O)N(CH₃)—CH═CH₂ (N-vinyl-N-methyl acetamide (VMA)).

Preferably, constituent A) is 2-hydroxyethylmethacrylate (HEMA) or those illustrated in the examples given hereunder.

Constituent A) may be present at a level of about 0.10 to 99.90% by weight in the composition, for example between 10.00 and 99.00 or between 50.00 and 90.00%.

“Polyol” means, in the sense of the present invention, any organic compound having at least two alcohol groups.

“Oligoglycerol” means, in the sense of the present invention, a polymer of glycerol, the average degree of polymerization of which is from 2 to 7, and moreover the oligoglycerol may be functionalized, with linear, branched or hyperbranched structure.

“Polyglycerol” means, in the sense of the present invention, a polymer of glycerol, the average degree of polymerization of which is greater than 7. It may for example be a polyglycerol having a molecular weight between 546 and 100 000 g/mol, preferably between 546 and 20 000 g/mol, and more particularly between 2000 and 5000 g/mol, 546 g/mol not being included.

The polyglycerol may be obtained by any method known by a person skilled in the art, for example by the polymerization of a monomer initiated by an initiator in the presence of a catalyst, with or without a solvent. The average degree of polymerization must be strictly greater than 7, for example from 8 to 270, for example between 15 and 70.

“Linear polyglycerol” means, in the sense of the present invention, a polymer of glycerol comprising a skeleton of glycerol residues bound linearly, each monomer unit only being bound chemically by an ether bond to two other monomer units.

“Branched polyglycerol” means, in the sense of the present invention, a polymer of glycerol consisting of glycerol units joined together by ether bonds, whose molecular weight may be between 546 and 100 000 g/mol, for example between 546 and 10 000 g/mol, or between 800 and 6000 g/mol, 546 g/mol not being included. The branched polyglycerol may have degrees of polymerization (DP) for example greater than or equal to 8, dispersity between 1.1 and 5, for example between 1.1 and 1.8 and degrees of branching (DB), according to the definition of Frey et al. (Acta Polym. 1997.48, 30 ([3])), between 0.05 and 0.3.

“Hyperbranched polyglycerol” means, in the sense of the present invention, a polymer of glycerol consisting of glycerol units joined together by ether bonds, whose molecular weight may be between 546 and 100 000 g/mol, for example between 546 and 10 000 g/mol, or between 800 and 6000 g/mol, 546 g/mol not being included. The hyperbranched polyglycerol may have degrees of polymerization (DP) for example greater than 8, dispersity between 1.1 and 5 and degrees of branching (DB) according to the definition of Frey et al. ([1]) between 0.3 and 0.8, and may even be up to 1.0 by doping the DB by post-polymerization modification. For example, the hyperbranched polyglycerol may be a polyglycerol dendrimer or a hyperbranched polyglycerol with a degree of branching between 0.3 and 0.7, or a mixture of hyperbranched polyglycerol and of oligoglycerols or of linear polyglycerols. The hyperbranched polyglycerol may be obtained by any technique known by a person skilled in the art, for example by the method described by Sunder et al. (“Controlled Synthesis of Hyperbranched Polyglycerols by Ring-Opening Multibranching Polymerization”, Macromolecules 1999, 32, 13, 4240-4246 ([5])) to obtain average molecular weights of up to 6000 g/mol and by the method described by Kainthan et al. (“Synthesis, Characterization, and Viscoelastic Properties of High Molecular Weight Hyperbranched Polyglycerols”, Macromolecules 2006, 39, 22, 7708-7717 ([6])) for obtaining average molecular weights above 6000 g/mol.

The number-average and weight-average molecular weights of the hyperbranched polyglycerol may be determined by size exclusion chromatography (SEC) and/or ¹H NMR spectroscopy.

In general, the degree of branching (DB) may be determined using the conventional methods of the prior art, for example by Inverse Gated ¹³C NMR.

“Dendrimer” means, in the sense of the present invention, a molecule consisting of one or more dendrons emanating from a single constituent unit, a dendron being a molecule having a single free valence or focal unit, exclusively comprising repeating constituent units of a dendritic and terminal nature, in which each path of the free valence (focal unit) to any one of the terminal units comprises the same number of repeating constituent units. A polyglycerol dendrimer is a monodisperse macromolecule of glycerol, whose form is reminiscent of that of the branches of a tree, and whose structure is symmetrical.

Constituent B) may be present at a level from 0.1 to 99.99% by weight of the crosslinkable composition.

The hyperbranched polyglycerol may be obtained by any method known by a person skilled in the art, for example by ring-opening polymerization of glycidol or else of glycerol carbonate, using one or more mono- or polyfunctional initiators, for example such as:
monohydric alcohols such as methanol, butanol, phenol and derivatives thereof, benzyl alcohol, 1-dodecanol, 1-tetradecanol, 1-hexadecanol, the monoalkyl ether glycols such as glycol monoethyl ether or polyethylene glycol monoalkyl ethers, for example such as polyethylene glycol monoethyl ether,
diols such as ethylene glycol, diethylene glycol, triethylene glycol, the polyethylene glycols, 1,2-propanediol, 1,3-propanediol, 1,2-butanediol, 1,3-butanediol, 2,3-butanediol, 1,2-pentanediol, 1,3-pentanediol, 1,4-pentanediol, 1,5-pentanediol, 2,3-pentanediol, 2,4-pentanediol, 1,2-hexanediol, 1,3-hexanediol, 1,4-hexanediol, 1,5-hexanediol, 1,6-hexanediol, 2,5-hexanediol, 1,2-heptanediol, 1,7-heptanediol, 1,8-octanediol, 1,2-octanediol, 1,9-nonanediol, 1,10-decanediol, 1,2-decanediol, 1,12-dodecanediol, 1,2-dodecanediol, 1,5-hexadiene-3,4-diol, the cyclopentanediols, the cyclohexanediols, inositol and derivatives thereof, (2)-methyl-2,4-pentanediol, 2,4-dimethyl-2,4-pentanediol, 2-ethyl-1,3-hexanediol, 2,5-dimethyl-2,5-hexanediol, 2,2,4-trimethyl-1,3-pentanediol, pinacol, dipropylene glycol, 1,4-butanediol, hexamethylene glycol, bisphenol A, bisphenol F, bisphenol S, diethylene glycol, triethylene glycol, dipropylene glycol, tripropylene glycol, the polyethylene glycols HO(CH₂CH₂O)ₙ—H or the polypropylene glycols HO(CH[CH₃]CH₂O)ₙ—H (where n is an integer greater than or equal to 4) or mixtures of two or more of the compounds presented above,
triols or polyols of functionality above 3 such as glycerol, diglycerol, triglycerol, tetraglycerol, trimethylolethane, trimethylolpropane, di-trimethylolpropane, sorbitol,
oligomers with hydroxyl terminations such as ethoxylated pentaerythritols and propoxylated pentaerythrithols, ethoxylated trimethylolpropanes and propoxylated trimethylolpropanes, ethoxylated or propoxylated glycerols, resulting from the reaction of ring-opening addition of ethylene oxide and/or propylene oxide to pentaerythritol, trimethylolpropane and glycerol. The degree of ethoxylation or of propoxylation respectively is usually between 0.1 and 10 ethylene oxide units or propylene oxide units respectively per OH function. The molecular weight is generally between 100 and 1000 g/mol. Ethoxylated trimethylolpropanes, ethoxylated glycerols and ethoxylated pentaerythritol may preferably be used,
“star” molecules having at least three arms comprising polyoxypropylene-polyoxyethylene blocks may also be used such as ethoxylated or propoxylated sorbitols and saccharides, degraded starch, polyvinyl alcohol.
initiators such as water, methylamine, ethylamine, propylamine, butylamine, dodecylamine, myristylamine, palmitylamine, stearylamine, aniline, benzylamine, ortho- or para-toluidine, α,β-naphthylamine, ammonia, ethylenediamine, propylenediamine, 1,4-butylenediamine, 1,2-, 1,3-, 1,4-, 1,5-, or 1,6-hexamethylenediamines, as well as o-, m- and p-phenylenediamines, 2,4- and 2,6-tolylenediamine, 2,2′-, 2,4 and 4,4′-diaminodiphenylmethane, 2,2′-, 2,4 and 4,4′-diaminodicyclohexylmethane, diethylene glycol diamine, diethylene triamine, triethylene tetramine, bifunctional or trifunctional poly(propylene glycol)diamines (Jeffamines),
amino alcohols such as diethanolamine, dipropanolamine, diisopropanolamine, triethanolamine, tris(hydroxymethyl)aminomethane or diisopropylethanolamine,
compounds containing functional groups such as allyl alcohol, allylglycerol, 10-undeceneol, 10-undeceneamine, dibenzylamine.

The initiator may then be partially deprotonated with a suitable agent, for example selected from alkali metals and their hydrides, alkoxides, and hydroxides. Preferably, the metals or the alkoxides of metals such as potassium methoxide (MeOK) are used. Potassium carbonate may also be used as a catalyst of the polymerization of glycerol carbonate in particular.

Other molecules may be used for catalyzing the polymerization, for example such as alcoholates, organometallics, metal salts, and tertiary amines. Among the alcoholates, the alcoholates of alkali metals will be used, such as sodium methylate, potassium isopropylate, potassium methoxide. Tetraalkylammonium hydroxides may also be used, such as tetramethylammonium hydroxides; hydroxides of alkali metals, such as sodium hydroxide and potassium hydroxide, metal salts, such as organic and/or inorganic compounds based on iron, lead, bismuth, zinc and/or tin at conventional levels of oxidation of metals, for example: iron(II) chloride, iron(III) chloride, bismuth(III) 2-ethylhexanoate, bismuth(III) octoate, bismuth(III) neodecanoate, zinc chloride, zinc 2-ethylcaproate, tin(II) octoate, tin(II) ethylcaproate, tin(II) palmitate, tin(IV) dibutyldilaurate (DBTL), tin(IV) dibutyldichloride or lead octoate; amidines, such as 2,3-dimethyl-3,4,5,6-tetrahydropyrimidine.

Alkali metal salts of long-chain fatty acids having 10 to 20 carbon atoms and optionally pendent OH groups may also be used. Finally, the tertiary amines such as triethylamine, tributylamine, dimethylbenzylamine, diethylbenzylamine, pyridine, methylpyridine, dicyclohexylmethylamine, dimethylcyclohexylamine, N,N,N′,N′-tetramethyldiaminodiethyl ether, bis(dimethylaminopropyl)-urea, N-methyl- and N-ethylmorpholine, N-cocomorpholine, N-cyclohexylmorpholine, N,N,N′,N′-tetramethylethylenediamine, N,N,N′,N′-tetramethyl-1,3-butanediamine, N,N,N′,N′-tetramethyl-1,6-hexanediamine, pentamethyldiethylenetriamine, N-methylpiperidine, N-dimethylaminoethylpiperidine, N,N′-dimethylpiperazine, N-methyl-N′-dimethylaminopiperazine, 1,8-diazabicyclo(5.4.0)undec-7-ene (DBU), TBD (1,5,7-triazabicyclo[4.4.0]dec-5-ene), 1,2-dimethylimidazole, 2-methylimidazole, N,N-dimethylimidazole-[3-phenylethylamine, DABCO or 1,4-diazabicyclo-(2,2,2)octane, bis(N,N-dimethylaminoethyl)adipate; compounds of the alkanolamine type, such as triethanolamine, triisopropanolamine, N-methyl- and N-ethyl-diethanolamine, dimethylaminoethanol, 2-(N,N-dimethylaminoethoxy)ethanol, the N,N′,N″-tris-(dialkylaminoalkyl)hexahydrotriazines, such as N,N′,N″-tris-(dimethylaminopropyl)-s-hexahydrotriazine and/or (dimethylaminoethyl)ether.

The reaction may take place in the presence of a solvent, for example an aliphatic, cycloaliphatic or aromatic solvent such as Decalin, toluene or xylene, or an ether such as glyme, diglyme or triglyme). Alternatively, the reaction may take place in the bulk, for example between 40 and 140° C., preferably at 95° C. in semibatch mode, by controlled, slow addition of the monomers to the reaction medium.

The hyperbranched polyglycerol can also be obtained by co-polymerization with other functionalized monomers that may incorporate at least one group selected from fluorinated groups, silanes, siloxanes, and halogenated compounds such as propylene oxide, ethylene oxide, butylene oxide, epichlorohydrin, vinyloxirane, glycidyl allyl ether, glycidyl methacrylate, isopropyl glycidyl ether, phenylglycidyl ether, 2-ethylhexyl glycidyl ether, hexadecyl glycidyl ether, naphthyl glycidyl ether, t-butyldimethylsilyl-(R)-(−)-glycidyl ether, benzyl glycidyl ether, epoxy-3-phenoxypropane, biphenylyl glycidyl ether, propargyl glycidyl ether, the n-alkyl-glycidyl ethers, but also functionalized oxiranes such as γ-glycidylpropyltrimethoxysilane, γ-glycidylpropyltriethoxysilane, γ-glycidoxypropyl-bis(trimethylsiloxy)-methylsilane and 3-(bis(trimethylsiloxy)methyl)-propyl glycidyl ether, glycidyl glycerol ether, glycidyl butyl ether, glycidyl nonylphenyl ether, the fluorinated and perfluorinated oxiranes such as hexafluoropropylene oxide, 2,3-difluoro-2,3-bis-trifluoromethyl-oxirane, 2,2,3-trifluoro-3-pentafluoroethyl-oxirane, 2,3-difluoro-2-(1,2,2,2-tetrafluoro-1-trifluoromethyl-ethyl)-3-trifluoromethyl-oxirane, 2-fluoro-2-pentafluoroethyl-3,3-bis-trifluoromethyl-oxirane, 1,2,2,3,3,4,4,5,5,6-decafluoro-7-oxa-bicyclo[4.1.0]heptane, 2,3-difluoro-2-trifluoromethyl-3-pentafluoroethyl-oxirane, 2,3-difluoro-2-nonafluorobutyl-3-trifluoromethyl-oxirane, 2,3-difluoro-2-heptafluoropropyl-3-pentafluoroethyl-oxirane, 2-fluoro-3-pentafluoroethyl-2,3-bis-trifluoromethyl-oxirane, 2,3-bis-pentafluoroethyl-2,3-bistrifluoromethyl-oxirane, 2,3-bis-trifluoromethyl-oxirane, 2-pentafluoroethyl-3-trifluoromethyl-oxirane, 2-(1,2,2,2-tetrafluoro-1-trifluoromethyl-ethyl)-3-trifluoromethyl-oxirane, 2-nonafluorobutyl-3-pentafluoroethyl-oxirane, 2,2-bis-trifluoromethyl-oxirane, 2-heptafluoroisopropyloxirane, 2-heptafluoropropyloxirane, 2-nonafluorobutyloxirane, 2-tridecafluorohexyloxirane, 2-pentafluoroethyl-2-(1,2,2,2-tetrafluoro-1-trifluoromethyl-ethyl)-3,3-bis-trifluoromethyl-oxirane, 2-fluoro-3,3-bis(1,2,2,2-tetrafluoro-1-trifluoromethyl-ethyl)-2-trifluoromethyl-oxirane, 2-fluoro-3-heptafluoropropyl-2-(1,2,2,2-tetrafluoro-1-trifluoromethyl-ethyl)-3-trifluoromethyl-oxirane and 2-(1,2,2,3,3,3-hexafluoro-1-trifluoromethyl-propyl)-2,3,3-tris-trifluoromethyl-oxirane.

These types of compounds or groups may be introduced in situ by copolymerization or by post-modification.

Preferably, the polyglycerol is a hyperbranched polyglycerol.

Constituent B), especially when it is a functionalized hyperbranched polyglycerol, may be present in the composition as crosslinking agent, as comonomer or else as principal monomer. Once functionalized, for example with vinylic or acrylic reactive functions such as acrylate, methacrylate functions, in particular the isocyanatoalkyl(meth)acrylates such as 2-isocyanatoethylmethacrylate and 2-isocyanatoethyl acrylate, maleate, fumarate, itaconate, citraconates and reactive vinyls, it may be used as a crosslinking agent. The functional monomers may also allow improvement of the physicochemical properties of the material such as monomers of siloxane, silane, and fluorinated types.

The polymerizable C═C function on the functionalized hyperbranched polyglycerol may be selected from the acrylic, methacrylic, maleic, fumaric, itaconic, or vinylic groups of structure:

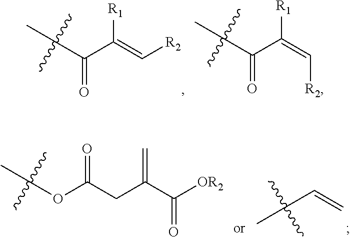

in which R₁ represents H or methyl; R₂ represents H, linear or branched C₁₋₆alkyl, or —C(═O)OR_{2A} in which R_{2A} represents H or linear or branched C₁₋₆alkyl optionally substituted with one or more hydroxyl groups, and R₃ represents H, linear or branched C₁₋₂₀alkyl.

The group bearing a polymerizable C═C function on the functionalized hyperbranched polyglycerol may be selected from:

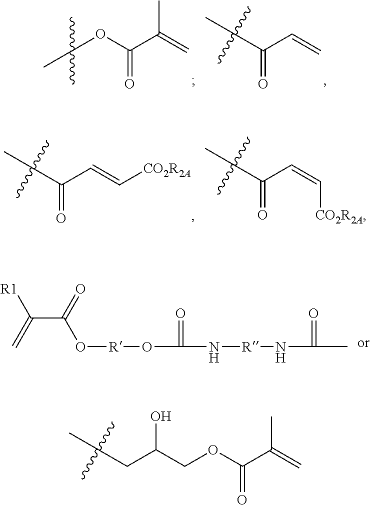

where:
R₁═H or CH₃
R_{2A} represents H or linear or branched C₁₋₆alkyl substituted with one or more hydroxyl groups, preferably

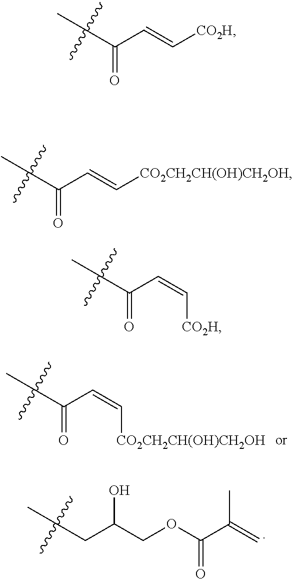

R′ and R″ represent, independently, a linear or branched alkylene radical, saturated or unsaturated, substituted or unsubstituted, having from 2 to 20 carbon atoms, a saturated or unsaturated, substituted or unsubstituted cycloalkylene radical, having from 3 to 20 carbon atoms, a substituted or unsubstituted arylene radical having from 6 to 20 carbon atoms, an arylene-alkylene radical having from 7 to 20 carbon atoms, a heterocyclic radical or any linear or branched sequence of two or more of the radicals mentioned, optionally joined together via an ether, thioether, ester, amine or amide structure.

The functionalization may be done by any method known by a person skilled in the art, for example in semibatch mode by slow, controlled addition of the functionalizing agent and in the presence or absence of a polar solvent, under inert gases, preferably at a temperature between 25 and 200° C. and more particularly between 60 and 90° C. In order to be used as a crosslinking agent, the polyol, for example polyglycerol, must have an average functionalization greater than 1, preferably greater than or equal to 2, and may be up to 100% by number of OH present on the hyperbranched polyglycerol molecule.

For example, pure polyglycerol or the copolymer of glycidol and another oxirane monomer may be functionalized by a reaction of addition or condensation on the OH functions with an agent selected from:
maleic anhydride and derivatives of maleic acid such as maleates and fumarates. It may be for example dimethyl maleate, diethyl maleate, or higher maleate esters where R is an alkyl group containing 3 to 20 carbon atoms, fumaric acid, dimethyl fumarate, diethyl fumarate or higher fumarate esters where R is an alkyl group containing 3 to 20 carbon atoms. The reaction of functionalization with the anhydride may be carried out between 0° C. and 120° C. The reaction with maleic acid or the maleates or fumarates may be carried out at a temperature between 90° C. and 200° C. in the presence or absence of esterification or transesterification catalysts and in conditions familiar to a person skilled in the art,
itaconic anhydride and derivatives thereof, such as itaconic acid, dimethyl itaconate, or the higher itaconate esters, where R is an alkyl group containing 3 to 20 carbon atoms. The reaction of functionalization with the anhydride may be carried out between 0° C. and 120° C. The reaction with itaconic acid or the itaconates may be carried out at a temperature between 90° C. and 200° C., in the presence or absence of esterification or transesterification catalysts and in conditions familiar to a person skilled in the art,
citraconic anhydride and derivatives thereof such as citraconic acid, dimethyl citraconate, or the higher citraconate esters, where R is an alkyl group containing 3 to 20 carbon atoms. The reaction of functionalization with the anhydride is carried out between 0° C. and 120° C. The reaction with citraconic acid or the citraconates is carried out at a temperature between 90° C. and 200° C., in the presence or absence of esterification or transesterification catalysts and in conditions familiar to a person skilled in the art,
isocyanatoalkyl(meth)acrylates, such as 2-isocyanatoethylmethacrylate, 2-isocyanatoethylacrylate. These isocyanato-alkyl(meth)acrylates are commercially available, or can also be synthesized according to the methodology described in document US20010005738 ([1]). Functionalization of the polyglycerol with an isocyanatoalkyl(meth)acrylate leads to the following group:

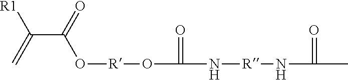

where:
R1═H or CH₃
R′ and R″ represent, independently, a linear or branched alkylene radical, saturated or unsaturated, substituted or unsubstituted, having from 2 to 20 carbon atoms, a cycloalkylene radical, saturated or unsaturated, substituted or unsubstituted, having from 3 to 20 carbon atoms, a substituted or unsubstituted arylene radical having from 3 to 20 carbon atoms, an arylenealkylene radical having from 4 to 20 carbon atoms, a heterocyclic radical or any linear or branched sequence of two or more of the radicals mentioned, optionally joined together via an ether, thioether, ester, amine or amide structure.

Preferably, in constituent B), the polymerizable C═C function of the acrylate or methacrylate type is selected from acrylic acid, methacrylic acid, the alkyl (meth)acrylates and derivatives thereof, such as 2-hydroxyethylmethacrylate (HEMA), 2-hydroxyethylacrylate (HEA), methylmethacrylate (MMA), methacrylamide, N,N-dimethyl-diacetoneacrylamide, 2-phosphatoethylmethacrylate, mono-, di-, tri-, tetra-, penta-polyethylene glycol acrylates or methacrylates, N-(3-methacrylamidopropyl)-N,N-dimethylamine, N-(3-methacrylamidopropyl)-N,N,N-trimethylamine, N-(3-acrylamido-3-methylbutyl)-N,N-dimethylamine, N-methacrylamide, 3-hydroxypropyl methacrylate, propyl methacrylate, propyl acrylate, butyl methacrylate, butyl acrylate, pentyl acrylate, pentyl methacrylate, N-(1,1-dimethyl-3-oxobutyl)acrylamide, 2-ethyl-2-(hydroxy-methyl)-1,3-propanediol trimethacrylate, butyl(meth)acrylate, 2-hydroxybutyl methacrylate, 3-hydroxy-2-naphthyl methacrylate, N-(formylmethyl)acrylamide, 2-ethoxyethyl methacrylate, 4-t-butyl-2-hydroxycyclohexyl methacrylate, the isocyanatoalkyl(meth)acrylates such as 2-isocyanatoethylmethacrylate, 2-isocyanatoethyl acrylate, and a compound selected from 4-[(a)-phenyldiazenyl]phenyl-2-methacrylate (4-[(E)-phenyldiazenyl]phenyl-2-methacrylate) and 4-[(a)-phenyldiazenyl]phenyl-2-methacrylate (4-[(E)-phenyldiazenyl]phenyl-2-methacrylate). Preferably, it is an isocyanatoalkyl(meth)acrylate, for example selected from 2-isocyanatoethylmethacrylate and 2-isocyanatoethyl acrylate, and a mixture thereof.

In the case of the anhydrides that generate an acid function after reaction with an OH function of the polyglycerol, or in the case of functionalization with a diacid, the residual carboxylic acid function may then react with an epoxy function of a compound of the epoxysilane, epoxyfluorinated, glycidyl(meth)acrylate, monoepoxy type such as glycidol, the oxiranes usable as comonomers as defined above, or any other monoepoxy of formula:

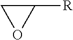

This reaction leads to the formation of an ester bond by reaction on the carboxylic acid function. The nature of the radical R will allow the properties of the polyol, in particular of the polyglycerol, to be adjusted.

Alternatively, Williamson etherification reactions may be implemented by reaction of allyl halides on hydroxyl functions, but also by alkylation by phase transfer in the presence of sodium hydroxide as described in Journal of the American Society (2000) 122, 2954-2955 ([4]).

In another embodiment, the hyperbranched polyglycerol functionalized with at least one unsaturation, preferably 2 unsaturations, may be additionally functionalized with groups selected from fluorinated groups, silicones and silanes, by reaction of monomers as described above in relation to constituent A). In this embodiment, it may be a direct reaction of the epoxy function on the OH end groups, either before, or after functionalization with unsaturated groups. Alternatively, it may be a reaction on the carboxylic acid function generated by the reaction of functionalization with the maleic, itaconic and citraconic anhydrides in particular.

Regarding constituent C), this initiator may be a UV, thermal or redox initiator, such as:
a photochemical initiator of the hydroxybenzophenone class such as 2-hydroxy-4-acryloyloxyethoxybenzophenone, 2-hydroxy-4-(2-hydroxy-3-methacrylyloxy)propoxybenzophenone; benzophenone, acetophenone, acetonaphthoquinone, methyl ethyl ketone, valerophenone, hexanophenone, C-phenylbutyrophenone, p-morpholinopropiophenone, dibenzosuberone, 4-morpholinobenzophenone, 4-morpholinodeoxybenzoin, p-diacetylbenzene, 4-aminobenzophenone, para-methoxyacetophenone, 3-methylanthraquinone, tert-butylanthraquinone, the esters of anthraquinone, benzaldehyde, 9-acetylphenanthrene, 2-acetylphenanthrene, 10-thioxanthenone, 3-acetylphenanthrene, 3-acetylindole, 9-fluorenone, 1-indanone, 1,3,4-triacetylbenzene, thioxanthen-9-one, xanthen-9-one, 2,4-dimethylthioxanthone, 2,4-diethylthioxanthone, 2,4-diisopropylthioxanthone, 2,4-dichlorothioxanthone, benzoin, benzoin isobutyl ether, benzoin tetrahydropyranyl ether, benzoin methyl ether, benzoin ethyl ether, benzoin butyl ether, benzoin isopropyl ether, chloroxanthenone, 7-H-benzoin methyl ether, benzodeanthracen-7-one, 1-naphthaldehyde, 4,4′-bis(dimethylamino)benzophenone, 4-phenylbenzophenone, 4-chlorobenzophenone, 4,4′-bis(dimethylamino)benzophenone, 1-acetonaphthone, 2-acetonaphthone, 1-benzoylcyclohexan-1-ol, 2-hydroxy-2,2-dimethylacetophenone, 2,2-dimethoxy-2-phenylacetophenone, 2,2-diethoxy-2-phenylacetophenone, 1,1-dichloroacetophenone, 1-hydroxyacetophenone, acetophenone dimethyl acetal, o-methoxybenzophenone, triphenylphosphine, tris-o-tolylphosphine, benzaanthracene-7,12-dione, 2,2-diethoxyacetophenone, the benzyl acetals such as benzyl dimethyl acetal, 2-methyl-1-4-(methylthio)phenyl-2-morpholinopropan-1-one, the anthraquinones such as 2-methylanthraquinone, 2-ethylanthraquinone, 2-tert-butylanthraquinone, 1-chloroanthraquinone, 2-amylanthraquinone, 2,3-butanedione,
photoinitiators causing little or no yellowing such as esters of phenylglyoxalic acid, combinations of the various photoinitiators as mentioned above, and the photoinitiators in combination with photopolymerization promoters such as benzoic acid or amines,
a thermal initiator of the nitrile type such as 2,2-azobis(2,4-dimethylpentanenitrile), 2,2′-azobisisobutyronitrile, 1,1′-azobis(cyclohexanecarbonitrile), or 2,2′-azobis(2-methylpropionitrile), a thermal initiator of the peroxide type such as potassium peroxodisulfate, dibenzoyl peroxide, cyclohexanone peroxide, di-tert-butyl peroxide, acetyl cyclohexylsulfonyl peroxide, diisopropyl percarbonate, tert-butyl peroctoate or benzopinacol, di-t-butyl peroxide, cumene hydroperoxide, dicumyl peroxide, t-butyl perbenzoate, or a hydroxylated N-oxide amine, such as 2,2,6,6-tetramethylpiperidine-N-oxyl and 4-hydroxy-2,2,6,6-tetramethylpiperidine-N-oxyl,
a redox initiator system allowing the polymerization reactions to be carried out at low temperature, such as oxidants including the azo derivatives such as AIBN, peroxides for example such as hydrogen peroxide, benzoyl peroxide, t-butyl-hydroperoxide, p-mentane hydroperoxide, ammonium persulfate, sodium persulfate, potassium persulfate, reducing agents such as ascorbic acid, sodium bisulfite, N,N,N′,N′-tetramethylethylene diamine formaldehyde, sodium sulfoxylate, the Fe²⁺, Cr²⁺, Zn²⁺, V²⁺, Ti³⁺, CO₂⁺, Cu⁺ metal salts for example such as iron sulfate FeSO₄ in particular, as well as the N,N-dialkylanilines such as N,N-dimethyl-p-toluidine.

The filtering agents may be selected from the filters for protecting the eye against radiation of the visible region or near-visible region such as UV filters, UV absorbents, or blue light filters, photochromic compounds, and infrared filters or filters of visible light with specific absorption bands. The filtering agents suitable for use with the crosslinkable composition according to the invention may be any suitable filtering agent that is commercially available, such as filters for protecting the eye against radiation in the visible region or near-visible region such as UV filters, UV absorbents, or blue light filters, photochromic compounds, and infrared filters or filters of visible light with specific absorption bands. It may be for example AEHB (acryloxyethoxyhydroxybenzophenone). Moreover, the UV absorbents may be any UV absorbent having high absorption in the UV-A range (320-380 nm), but relatively transparent beyond 380 nm. Generally, if the UV absorbent is present in the composition of the invention, it is between 0.5 and 1.5% by weight of the reactants, for example at 1% by weight. Advantageously, the anti-UV agents may be incorporated in the hydrogel in the post-polymerization hydration step.

The invention further relates to a crosslinked composition able to form a hydrogel polymer, resulting from the crosslinking of:

A) at least one polymerizable monomer or a mixture of polymerizable monomers or a mixture of monomers and polymerizable macromonomers bearing at least one polymerizable C═C function of the acrylate, methacrylate, maleate, fumarate, itaconate, citraconate, styrenic or vinylic type;

B) at least one linear, branched or hyperbranched polyglycerol or a mixture of hyperbranched polyglycerol and of linear, branched or hyperbranched polyglycerols comprising on average more than 1 hydroxyl group functionalized with a group bearing a polymerizable C═C function of the acrylate, methacrylate, maleate, fumarate, itaconate, citraconate, styrenic or vinylic type;

C) a radical initiator or a mixture of radical initiators capable of initiating polymerization by thermal, redox or photochemical routes; and

D) optionally at least one additive selected from antioxidants, agents for adjusting the physicochemical properties such as Young's modulus, elongation at break and breaking stress, agents for promoting, modulating and/or controlling permeability to oxygen, plasticizers, moistening agents, lubricants, viscosity modifiers, compatibilizers, colorants, filtering agents, therapeutic agents, antimicrobial agents and agents against bacterial biofilms.

The constituents A), B), C) and D) are as described above in the context of the crosslinkable composition.

The formation of a gel may be obtained with a crosslinked composition of the invention, comprising constituent A), constituent B) at a level of 0.10 to 99.99% by weight of the crosslinked composition and constituent C) present between 0.05 and 5% by weight.

The invention further relates to a hydrogel obtainable by:
hydration/swelling, from water or an aqueous solution, of a crosslinked composition as defined above, or,
polymerization of the crosslinkable composition in the presence of a water-miscible protic or aprotic solvent, then exchange of the solvent with an excess of water or an aqueous solution, or
by polymerization of the crosslinkable composition in the presence of water or an aqueous solution.

The hydration/swelling step may be carried out by a person skilled in the art for a time that is adjusted to the swelling required. It may be carried out for example for 12 h, at room temperature (i.e. about 25° C.).

Advantageously, the aqueous solution may contain a compound of interest selected from therapeutic active ingredients, vitamins or lubricants. They may be for example tocopherols, which are very powerful antioxidants for treating chronic anterior uveitis, or hyaluronic acid, for treating or preventing ocular dryness.

The invention further relates to a method for preparing a crosslinked composition of the invention, consisting of reacting, in the presence or absence of aprotic solvent, preferably in the absence of solvent:

A) at least one polymerizable monomer or mixture of polymerizable monomers or a mixture of monomers and polymerizable macromonomers bearing at least one polymerizable C═C function of the acrylate, methacrylate, maleate, fumarate, itaconate, citraconate, styrenic or vinylic type;

B) at least one polyol selected from oligoglycerols, linear, branched and hyperbranched polyglycerols, or a mixture of hyperbranched polyglycerol and of a polyol selected from oligoglycerols and linear, branched and hyperbranched polyglycerols, comprising on average more than 1 hydroxyl group functionalized with a group bearing a polymerizable C═C function of the acrylate, methacrylate, maleate, fumarate, itaconate, citraconate, styrenic or vinylic type;

C) at least one radical initiator or a mixture of radical initiators that are able to initiate polymerization by thermal, redox or photochemical routes; and

D) optionally at least one additive selected from antioxidants, agents for adjusting the physicochemical properties such as Young's modulus, elongation at break and breaking stress, promoters of permeability to oxygen, plasticizers, moistening agents, lubricants, viscosity modifiers, compatibilizers, colorants, filtering agents, antimicrobial agents, therapeutic agents and agents against bacterial biofilms. The constituents A), B), C) and D) are as described above in the context of the crosslinkable composition.

In this method:
the aprotic solvent, when it is used, may be selected from the polar aprotic solvents such as chloroform, dimethylformamide, dichloromethane, acetonitrile, and dimethylsulfoxide, or a mixture of at least two of them,
the protic solvent, when it is used, may be selected from water, methanol and ethanol.

In this method, constituent B), especially when it is a functionalized hyperbranched polyglycerol, may be present at a rate from 0.1 to 99.99% by weight of the total weight of constituents A) to D).

In this method, the initiator may be present at a rate from 0.02 to 5% by weight, preferably 0.02 to 2%, of the total weight of constituents A) to D).

Advantageously, this method may further comprise a step of molding the crosslinked composition. The molding step may be carried out by molding by casting, block molding in order to be machined by cutting by turning, centrifugal casting or additive manufacturing, preferably in an inert atmosphere.

The method may be carried out at a temperature from 0° C. to 150° C., preferably from 0° C. to 120° C. Within this temperature range, the lowest temperatures, i.e. from 0° C. to about 60° C., relate to redox systems. The photopolymerizations take place at room temperature or a little higher, for example between 15° C. and 39° C. Starting from 40° C., thermal initiation systems may be used.

The method may further comprise an annealing step, preferably carried out at a temperature from 20 to 200° C., preferably from 60 to 160° C. This step may be carried out for a suitable time, for example in the range from 1 minute to 48 h.

The method may further comprise a step of:
hydration/swelling, from water or an aqueous solution, of a crosslinked composition according to the invention, or,
polymerization of the crosslinkable composition according to the invention in the presence of a water-miscible protic or aprotic solvent, then exchange of the solvent with an excess of water or an aqueous solution, or
polymerization of the crosslinkable composition according to the invention in the presence of water or an aqueous solution.

As stated above, the aqueous solution may contain a compound of interest selected from therapeutic active ingredients, vitamins, nutrients, decontaminating agents or lubricants, for example such as hyaluronic acid.

The invention further relates to an article obtainable by a method for preparing a crosslinked composition as defined above. The article may be a medical device, such as a contact lens or intraocular lens, a patch, an implant or a dressing.

The invention further relates to the use of a composition or of a hydrogel according to the invention as defined above, for manufacturing a medical device, such as a contact lens or intraocular lens, a patch, an implant or a dressing.

The invention further relates to the use of a composition or of a hydrogel according to the invention as defined above, as a vector of a compound of interest selected from therapeutic active ingredients, vitamins, nutrients, decontaminating agents or lubricants.

The invention further relates to the use of a hyperbranched polyglycerol comprising on average more than one group(s), preferably at least 2 hydroxyl groups functionalized with a group bearing a polymerizable C═C function, as crosslinking agent, comonomer or else principal monomer for manufacturing a contact lens or intraocular lens.

The invention further relates to the use of a composition or of a hydrogel according to the invention, in biomedical, cosmetic, or health-related applications. They may be for example applications in surgical devices, release of active ingredients, tissue engineering, dressings, culture of organs, biological tissues, water reservoirs for plants, perfume diffusers, depolluting agents, culture media for bacteria, and absorbents of liquids in diapers for babies, feminine hygiene products and absorbent products for incontinence.

Among the active ingredients able to be released by the composition or the hydrogel of the invention, we may mention any known active ingredient, for example all classes of anti-inflammatories, such as NSAIDs or corticoids, all classes of antibiotics, singly or combined, all classes of antiglaucoma drugs, singly or combined, all classes of antiallergic drugs, singly or combined, all classes of drugs for treating progression of myopia, such as anticholinergics, drugs for treating presbyopia, and more generally treatments of ocular disorders including the eye as a whole and its appendages, namely the eyelids, the oculomotor muscles, the lacrimal glands and its secretions and the orbits.

Among the nonmedicinal substances able to be released by the composition or hydrogel of the invention, we may mention for example vitamins, nutrients such as antioxidants, protectors of the metabolism or the agents for decontaminating the lens, such as agents against bacterial biofilms, antifungals, amebicides or antivirals.

Other advantages may also be evident to a person skilled in the art on reading the following examples, given for purposes of illustration.

### EXAMPLES

### List of Acronyms

### AIBN: Azobisisobutyronitrile

HPG: Hyperbranched polyglycerol

### TMP: Trimethylolpropane

MeOK: Potassium methoxide
MA: Maleic anhydride
IA: Itaconic anhydride

### IEA: Isocyanatoethylacryate

f: functionality of a molecule, i.e. average number of reactive functions per (macro)molecule
% f: molar percentage of functionalized OH per (macro)molecule

### DMF: N,N-Dimethylformamide

### HEMA: 2-Hydroxyethylmethacrylate

EWC: equilibrium water content by weight in the gel as a percentage (%)

### Determination of the Properties Employed in the Examples

Determination of Permeability to Oxygen (Dk) by Polarography

The polarographic method is based on a conventional setup in electrochemistry comprising 3 electrodes: working electrode (WE) made of gold, counter electrode (CE) made of platinum, and Ag/AgCl reference electrode (RE), immersed in a solution of electrolyte (KCl) at 0.1 M. The hydrogel is placed on the surface of the working electrode and then oxygen is injected into the electrochemical cell and the variation of current is measured (oxidation of the oxygen at the surface of the WE). The measured current intensity will depend on the amount of oxygen that has passed through the hydrogel.

Three tests are performed for each sample, and the average of the three analyses is retained.

The potentiostat used for these analyses is a DropSens pSTAT 400.

### Determination of the Mechanical Properties

Using a tensile tester (M500-30AT) equipped with a DBBMTCL 50 kg Testometric cell, the mechanical properties determined are: Young's modulus, breaking stress and elongation at break.

The dimensions of the hydrated samples are standardized at width of 3 mm for 10 mm between the jaws; the thickness is measured at each new analysis. The preload is 0.1N for a speed of deformation equivalent to 20 mm/min, the whole at room temperature. All the samples were evaluated at least 3 times and mean values of the data calculated with WinTest software were calculated.

### Determination of the Surface Properties

The materials developed are also analyzed by various techniques including measurements of wettability (a), of surface roughness by atomic force microscopy (AFM) (b), and of coefficients of friction using the tribometer (c)

(a) Measurements of Wettability

The surfaces properties were also determined by measurements of contact angles of water on the materials using the drop-on-solid method on KRUSS DSA 100 apparatus. Briefly, a drop (2 μL) of distilled water is deposited on the surface of the material and the angle (in °) at equilibrium of the drop with the material is measured by means of a video camera. A mean value from 10 measurements is obtained using DropShapeAnalysis software.

(b) Measurements of Surface Roughness by Atomic Force Microscopy (AFM)

The AFM analyses were carried out on Dimension EDGE equipment from Bruker. The analyses were carried out in Tapping mode. Force levers of 3 N/m with Si₃N₄ tips (Bruker, Ref: RFESP) were used for generating the images of phase, amplitude and height. The photographs of height enabled us to find R(max), Ra and Rq, defined respectively as the maximum height identified on the surface of the sample; the mean surface roughness and the standard deviation from the mean flat surface. The samples were analyzed on lengths of 20, 10, 5 and 1 μm to generate scanned areas of 400, 100, 25 and 1 μm². The data were processed with Nanoscope Analysis software, and compared with commercial lenses.

(c) Measurement of Coefficients of Friction with the Tribometer

The measurements were carried out on a CSM instrument tribometer. A steel ball with a diameter of 10 mm was used at a speed of 1 cm/s, with a normal force of 0.5 N. 3 analyses were performed on each sample and the mean value of the 3 was calculated. The sample is in the form of film, and the analysis takes place in a liquid medium (water or normal saline solution) at room temperature.

### UV Transmittance Analyses

The transmittance was determined using a UV spectrophotometer. A lens is placed in a tank containing a saline solution. The tank is put in the sample compartment. A tank containing only saline solution is put in the reference compartment.

The spectrum in % transmittance is recorded between 200 and 780 nm. The sample is analyzed 3 times and the mean value of the 3 measurements at 550 nm was retained.

### Water Content and Degree of Swelling

The water content and the degree of swelling are determined by measuring the weight of the gel in the dry state and in the hydrated state using equations 1 and 2.

The gels in the hydrated state are weighed individually after removing excess water from the surface. The gels are then dried in a stove at 80° C. for a minimum of 6 h and weighed again. This process is repeated 3 times, and the value of EWC is the average of the 3.

### Example A: Synthesis of a Hyperbranched Polyglycerol with Mₙ=2487 g·mol⁻¹

Trimethylolpropane (TMP; 1 eq, 3.417 g) and potassium methoxide at 25% in methanol (MeOK; 0.3 eq, 2.126 g) are put in a 250 mL three-necked flask.

The flask is then put in a rotary evaporator at 70° C. until the TMP has dissolved completely, and then the rotary evaporator is put under vacuum to remove the methanol.

The three-necked flask containing the reactants is then put in an oil bath thermostatically controlled to 95° C., provided with a stirring paddle (300 rpm) under a nitrogen stream. Once the reactor is at temperature, glycidol (35.5 eq, 60.03 g) is added with a peristaltic feed pump at a rate of 3.6 mL/h.

Once addition of glycidol has ended, the reaction mixture is stirred for two hours at 95° C. to ensure complete conversion of the glycidol. The polymer obtained is dissolved in methanol, neutralized and de-ionized with Amberlite® and then precipitated twice in acetone.

The hyperbranched polyglycerol obtained is characterized by size exclusion chromatography (SEC) and ¹H NMR spectroscopy.

δ (ppm), MeOD: 4.92 (OH); 3.56 (CH₂—CH₂—O)ₙ₋₂; 1.36 (CH₂, TMP); 0.87 (CH₃, TMP)

### Example B: Synthesis of Hyperbranched Polyglycerol with Mₙ=2440 g·Mol⁻¹

The same protocol as in example A was used for producing a hyperbranched polyglycerol of average molecular weight 2440 g·mol⁻¹ by changing the proportions of the reactants in accordance with Table 1.

### Example C: Synthesis of Hyperbranched Polyglycerol with Mₙ=4037 g·Mol⁻¹

The same protocol as in example 1 was used for producing a polyglycerol of average molecular weight 4037 g·mol⁻¹ by changing the proportions of the reactants in accordance with Table 1.

### Example D: Synthesis of Macropolyol with Mₙ=4550 g·Mol⁻¹

The same protocol as in example 1 was used for producing a polyglycerol of average molecular weight 4550 g·mol⁻¹ by changing the proportions of the reactants in accordance with Table 1.

TABLE 1
Synthesis conditions of the hyperbranched polyglycerols

| | Mn | Mn | Weight | Weight | Weight |
|---|---|---|---|---|---|
| Examples of | theoretical | exp | TMP | MeOK | Glycidol |
| polyglycerols | (g/mol) | (g/mol) | (g) | (g) | (g) |
| A | 2487 | 2760 | 3.417 | 2.126 | 60.03 |
| B | 2440 | 2945 | 2.9 | 1.79 | 49.98 |
| C | 4037 | 4200 | 5.21 | 3.65 | 151.61 |
| D | 4550 | 5990 | 1.523 | 3.22 | 50.08 |

### Example 1: Functionalization of a Hyperbranched Polyglycerol (HPG) with Maleic Anhydride, for Obtaining a Functionalization f=9 Per HPG Macromolecule

The hyperbranched polyglycerol (HPG from example A: 2.128 g), previously purified and dried under vacuum, is put in a 125 mL two-necked flask.

The two-necked flask containing the reactant is then put in an oil bath thermostatically controlled to 80° C., provided with a stirring paddle (300 rpm) under a nitrogen stream. Once the reactor is at temperature, maleic anhydride (0.584 g) is added in one go.

The reaction is monitored by ¹H NMR spectroscopy and the reaction is stopped when there is no longer any maleic anhydride present in the reaction mixture.

### Examples 2 and 3: The Same Protocol is Used for Obtaining Different Functionalities by Changing the Proportions of Reactants in Accordance with Table 2

TABLE 2
Conditions for functionalization of hyperbranched
polyglycerol (HPG) with maleic anhydride

| | HPG | m_{HPG} | m_{maleic anhydride} | Functionality | % OH |
|---|---|---|---|---|---|
| Example | No. | (g) | (g) | f | functionalized |
| 1 | A | 2.128 | 0.584 | 9 | 22 |
| 2 | D | 2.092 | 0.550 | 14.5 | 18 |
| 3 | C | 2.1 | 0.710 | 14.5 | 25 |

with f: functionality of a molecule, i.e. average number of reactive functions per (macro)molecule

### Example 4: Functionalization of a Polyglycerol (HPG) with Itaconic Anhydride, for Obtaining a Functionalization f=2.5, Per HPG Macromolecule

Polyglycerol, previously purified and dried under vacuum, is put in a 125 mL two-necked flask (HPG from example B: 2.046 g)

The two-necked flask containing the reactant is then put in an oil bath thermostatically controlled to 110° C., provided with a stirring paddle (300 rpm) under a nitrogen stream. Once the reactor is at temperature, itaconic anhydride (0.291 g) is added in one go.

The reaction is monitored by ¹H NMR spectroscopy and the reaction is stopped when there is no longer any itaconic anhydride present in the reaction mixture.

Once the reaction has ended, the compound is dialyzed 3 times for 4 h in methanol, which is then evaporated under vacuum at room temperature.

The dry polymer is characterized by ¹H NMR spectroscopy.

### Example 5 to 11, 16 and 17: The Same Protocol is Used for Obtaining Different Functionalities by Changing the Proportions of Reactant in Accordance with Table 3

TABLE 3
Conditions for functionalization of HPG with itaconic anhydride

| | HPG | m_{HPG} | m_{itaconic anhydride} | Functionality | % OH |
|---|---|---|---|---|---|
| Example | No. | (g) | (g) | f | functionalized |
| 4 | B | 2.046 | 0.291 | 2.5 | 6 |
| 5 | | | | 2.5 | 6 |
| 6 | D | 2.224 | 0.212 | 3.3 | 4 |
| 7 | A | 2.177 | 0.649 | 5.6 | 15 |
| 8 | A | 2.035 | 0.295 | 2 | 5 |
| 9 | A | 2.465 | 1.206 | 10.4 | 27 |
| 10 | A | 1.943 | 0.282 | 2 | 5 |
| 11 | B | 1.95 | 0.188 | 1.7 | 4 |
| 16 | B | 2.4 | 0.179 | 2 | 4 |
| 17 | | 2.4 | 0.179 | | |

### Example 12: Functionalization of a Polyglycerol (HPG) with Isocyanatoethylacrylate, for Obtaining a Functionalization f=2, Per HPG Macromolecule

The polyglycerol, previously purified and dried under vacuum, is put in a 125 mL two-necked flask (HPG from example A: 2.046 g).

The two-necked flask containing the reactant is then placed at room temperature, provided with a stirring paddle (300 rpm) under a nitrogen stream. The catalyst, dibutyl tin laurate, (0.018 g) is added with 1.5 mL of DMF, previously distilled.

Once the mixture is homogeneous, the isocyanatoethylacrylate is added (0.271 g)

The reaction is monitored by ¹H NMR spectroscopy and the reaction is stopped when there is no longer any isocyanoethylacrylate present in the reaction mixture.

### Example 13 and 14: The Same Protocol is Used for Obtaining Different Functionalities by Changing the Proportions of Reactant in Accordance with Table 4

TABLE 4
Conditions for functionalization of
HPG with isocyanatoethylacrylate

| | HPG | m_{HPG} | m_{isocyanatoethylacrylate} | | % OH |
|---|---|---|---|---|---|
| Examples | No. | (g) | (g) | f | functionalized |
| 12 | A | 2.401 | 0.271 | 2 | 5 |
| 13 | A | 2.424 | 0.986 | 7 | 18 |
| 14 | C | 2.118 | 0.263 | 3.7 | 6 |

### Example A1: Method for Preparing Gels in Bulk Based on Polyglycerol Functionalized with Maleic Anhydride and 2-Hydroxyethylmethacrylate

The maleic anhydride functionalized polyglycerol (crosslinking agent) from example 1 (0.363 g, 36.3% by weight) is weighed in a bottle, and then 2-hydroxyethylmethacrylate (0.632 g, 63.2% by weight) and the thermal initiator: AIBN (0.005 g, 0.5% by weight) are added. The mixture is homogenized in a Speedmixer mixer for 30 s at 2500 rpm. Once homogenized, 0.2 mL of the mixture is introduced into a polypropylene mold using a micropipette. The mold is closed and then placed for 2 h in a stove preheated to 80° C. After 2 h, the material is removed from the mold while hot, before being characterized.

### Example A2 and A3: The Same Protocol is Used for Obtaining Formulations with Various Percentages by Weight of Polyglycerol with Maleic Functionality and HEMA, According to Table 5

TABLE 5
Formulations of gels based on HEMA and maleic
anhydride functionalized polyglycerol.

| | | | | % by weight | % by | % by |
|---|---|---|---|---|---|---|
| | No. HPG | | % OH | HPG | weight | weight |
| Example | functionalized | f | functionalized | functionalized | HEMA | AIBN |
| A1 | 1 | 9 | 22 | 36.3 | 63.2 | 0.5 |
| A2 | 2 | 14.5 | 18 | 8.3 | 81.2 | 0.5 |
| A3 | 3 | 14.5 | 18 | 22 | 77.5 | 0.5 |

Example A4: Method for Preparing Gels in Bulk Based on Itaconic Anhydride Functionalized Polyglycerol and 2-Hydroxyethylmethacrylate

The itaconic anhydride functionalized polyglycerol (crosslinking agent) from example 4 (0.281 g, 28.1% by weight) is weighed in a bottle, and then 2-hydroxyethylmethacrylate (0.714 g, 71.4% by weight) and the thermal initiator: AIBN (0.005 g, 0.5% by weight) are added. The mixture is homogenized in a Speedmixer mixer for 30 s at 2500 rpm. Once homogenized, 0.2 mL of the mixture is introduced into a polypropylene mold using a micropipette. The mold is closed and then placed for 2 h in a stove preheated to 80° C. After 2 h, the material is removed from the mold while hot, before being characterized.

### Example A5 to A11, A16: The Same Protocol is Used for Obtaining Formulations with Various Percentages by Weight of Itaconic Anhydride Functionalized Polyglycerol and HEMA, According to Table 6

### Example A17: Method for Preparing Gels in Bulk Based on Itaconic Anhydride Functionalized Polyglycerol, 2-Hydroxyethylmethacrylate and Methacrylic Acid

The itaconic anhydride functionalized polyglycerol (crosslinking agent) from example 4 (0.100 g, 10% by weight) is weighed in a bottle, and then 2-hydroxyethylmethacrylate (0.875 g, 85.7% by weight), methacrylic acid (comonomer) (0.02 g, 2%) and the thermal initiator: AIBN (0.005 g, 0.5% by weight), are added. The mixture is homogenized in a Speedmixer mixer for 30 s at 2500 rpm. Once homogenized, 0.2 mL of the mixture is introduced into a polypropylene mold using a micropipette. The mold is closed and then placed for 2 h in a stove preheated to 80° C. After 2 h, the material is removed from the mold while hot, before being characterized.

TABLE 6
Formulations of gels based on HEMA and polyglycerol functionalized with itaconic anhydride.

| | | | | % by | % by | % by | % by |
|---|---|---|---|---|---|---|---|
| | No. HPG | | % OH | weight HPG | weight | weight | weight |
| example | functionalized | f | functionalized | functionalized | HEMA | AIBN | methacrylic acid |
| A4 | 4 | 2.5 | 6 | 28.1 | 71.4 | 0.5 | / |
| A5 | 5 | 2.5 | 6 | 4.8 | 94.7 | 0.5 | / |
| A6 | 6 | 3.3 | 4 | 17.6 | 81.9 | 0.5 | / |
| A7 | 7 | 5.6 | 15 | 5 | 94.5 | 0.5 | / |
| A8 | 8 | 2 | 5 | 10 | 89.5 | 0.5 | / |
| A9 | 9 | 10.4 | 27 | 10 | 89.5 | 0.5 | / |
| A10 | 10 | 2 | 5 | 20 | 79.5 | 0.5 | / |
| A11 | 11 | 1.7 | 4 | 11.3 | 88.2 | 0.5 | / |
| A16 | 16 | 2 | 4 | 10 | 89.5 | 0.5 | / |
| A17 | 17 | 2 | 4 | 10 | 87.5 | 0.5 | 2 |

### Example A12: Method for Preparing Gels in Bulk Based on Isocyanatoethylacrylate Functionalized Polyglycerol and 2-Hydroxyethylmethacrylate

The isocyanatoethylacrylate functionalized polyglycerol (crosslinking agent) from example 12 (0.091 g, 9.1% by weight) is weighed in a bottle, and then 2-hydroxyethylmethacrylate (0.904 g, 90.4% by weight) and the thermal initiator: AIBN (0.005 g, 0.5% by weight), are added. The mixture is homogenized in a Speedmixer mixer for 30 s at 2500 rpm. Once homogenized, 0.2 mL of mixture is introduced into a polypropylene mold using a micropipette. The mold is closed and then placed for 2 h in a stove preheated to 80° C. After 2 h, the material is removed from the mold while hot, before being characterized.

### Example A13 and A14: The Same Protocol is Used for Obtaining Formulations with Various Percentages by Weight of Isocyanatoethylacrylate Functionalized Polyglycerol and HEMA, According to Table 7

TABLE 7
Various formulations of gels containing isocyanatoethylacrylate
functionalized polyglycerol and HEMA.

| | | | | % by weight | % by | % by |
|---|---|---|---|---|---|---|
| | No. HPG | | % OH | HPG | weight | weight |
| Example | functionalized | f | functionalized | functionalized | HEMA | AIBN |
| A12 | 12 | 2 | 5 | 9.1 | 90.4 | 0.5 |
| A13 | 13 | 7 | 18 | 4.5 | 95 | 0.5 |
| A14 | 14 | 3.7 | 6 | 4.8 | 94.7 | 0.5 |

### Example A15: Method for Preparing Gels in Bulk Based on 2-Hydroxyethylmethacrylate and Triethylene Glycol Dimethylmethacrylate (TEGDMA), Used as Reference

2-Hydroxyethylmethacrylate (0.990 g, 99% by weight) is weighed in a bottle, and then TEGDMA (0.005 g, 0.5% by weight) and the thermal initiator: AIBN (0.005 g, 0.5% by weight) are added. The mixture is homogenized in a Speedmixer mixer for 30 s at 2500 rpm. Once homogenized, 0.2 mL of mixture is introduced into a polypropylene mold using a micropipette. The mold is closed and then placed for 2 h in a stove preheated to 80° C. After 2 h, the material is removed from the mold while hot, before being characterized.

### Example A18: The Same Protocol is Used for Obtaining Formulations with Various Percentages by Weight of TEGDMA and HEMA, According to Table 8

TABLE 8
Various formulations of gels containing HEMA and TEGDMA

| | | % by weight | % by weight | % by weight |
|---|---|---|---|---|
| example | f | of TEGDMA | HEMA | AIBN |
| A15 | 2 | 0.5 | 99 | 0.5 |
| A18 | 2 | 0.5 | 99 | 0.5 |

TABLE 9
Water contents of the hydrogels (EWC in %) determined in
distilled water and in phosphate buffer solution (pH =
7.4, osmolarity = 280 mOsm)

| | EWC | EWC |
|---|---|---|
| Example | (water) | (phosphate buffer) |
| A1 | 71 | / |
| A2 | 53 | / |
| A3 | 44 | / |
| A4 | 61 | / |
| A5 | 52 | / |
| A6 | 57 | / |
| A7 | 43 | 44 |
| A8 | 50 | 50 |
| A9 | 43 | 48 |
| A10 | 50 | / |
| A11 | 44 | / |
| A12 | 49 | 46 |
| A13 | 42 | 42 |
| A15 | 41 | 39 |
| A18 | 40 | 41 |

TABLE 10
Variation, in the ambient air, of the water content
(EWC in %) as a function of exposure times of the gels
swollen with distilled water (Dimensions of the hydrogels
tested: diameter 23 mm; thickness 500 μm)

Retention in distilled water (EWC)

| example | 0 min | 30 min | 76 min | 131 min |
|---|---|---|---|---|
| A1 | 71 | 69 | 65 | 57 |
| A4 | 61 | 56 | 44 | 31 |
| A15 | 42 | 38 | 31 | 25 |
| A18 | 41 | 33 | 27 | 14 |

Retention in distilled water (EWC)

| example | 0 min | 15 min | 30 min | 60 min |
|---|---|---|---|---|
| A16 | 56 | 52 | 48 | 29 |
| A17 | 52 | 51 | 46 | 32 |
| A18 | 41 | 36 | 33 | 29 |

TABLE 11
Variation, in the ambient air, of the water content (EWC
in %) as a function of exposure times of the gels swollen
with phosphate buffer solution (Dimensions of the hydrogels
tested: diameter 23 mm; thickness 500 μm)

Retention in buffer solution (EWC)

| Examples | 0 min | 15 min | 30 min | 60 min |
|---|---|---|---|---|
| A16 | 51 | 49 | 44 | 28 |
| A17 | 59 | 56 | 52 | 39 |
| A18 | 40 | 36 | 32 | 15 |

TABLE 12
Time taken to reach a water loss of 10%
by weight (T₁₀) and 50% by weight (T₅₀)

| Examples | T₁₀ (min) | T₅₀ (min) |
|---|---|---|
| A1 | 82 | 251 |
| A4 | 30 | 128 |
| A18 | 20 | 98 |

TABLE 13
Mechanical properties of various formulations

| | Modulus | Breaking stress | Elongation at break |
|---|---|---|---|
| Examples | (MPa) | (MPa) | (%) |
| A3 | 0.558 | 1.035 | 240 |
| A6 | 0.893 | 0.608 | 269 |
| A7 | 0.58 | 0.788 | 319 |
| A10 | 0.566 | 0.558 | 314 |
| A12 | 0.94 | 1.475 | 358 |
| A13 | 1.11 | 0.769 | 164 |
| A15 | 0.772 | 0.798 | 333 |

TABLE 14
Permeability to oxygen

| Examples | Dk (barrer) |
|---|---|
| A3 | 21.3 |
| A11 | 12.3 |
| A13 | 12.4 |
| A15 | 15.8 |

### LIST OF REFERENCES

1. US20010005738.
2. WO2017150786.
3. Frey et al., Acta Polym. 1997, 48, 30.
4. Journal of the American Society (2000) 122, 2954-2955.
5. Sunder et al.: “Controlled Synthesis of Hyperbranched Polyglycerols by Ring-Opening Multibranching Polymerization”, Macromolecules 1999, 32, 13, 4240-4246.
6. Kainthan et al.: “Synthesis, Characterization, and Viscoelastic Properties of High Molecular Weight Hyperbranched Polyglycerols”, Macromolecules 2006, 39, 22, 7708-7717.

1. A crosslinkable composition comprising:
A) at least one polymerizable monomer or a mixture of polymerizable monomers or a mixture of monomers and polymerizable macromonomers bearing at least one polymerizable C═C function of the acrylate, methacrylate, maleate, fumarate, itaconate, citraconate, styrenic or vinylic type;
B) at least one polyol selected from oligoglycerols, linear, branched and hyperbranched polyglycerols, or a mixture of hyperbranched polyglycerol and of a polyol selected from the oligoglycerols, and the linear, branched and hyperbranched polyglycerols, comprising on average more than 1 hydroxyl group functionalized with a group bearing a polymerizable C═C function of the acrylate, methacrylate, maleate, fumarate, itaconate, citraconate, styrenic or vinylic type;
C) a radical initiator or a mixture of radical initiators that are able to initiate polymerization by thermal, redox or photochemical routes; and
D) optionally at least one additive selected from antioxidants, agents for adjusting the physicochemical properties, agents for promoting, modulating and/or controlling permeability to oxygen, plasticizers, moistening agents, lubricants, viscosity modifiers, compatibilizers, colorants, filtering agents, antimicrobial agents, therapeutic agents and agents against bacterial biofilms.

2. A crosslinked composition able to form a hydrogel polymer, resulting from the crosslinking:
A) at least one polymerizable monomer or mixture of polymerizable monomers or a mixture of monomers and polymerizable macromonomers bearing at least one polymerizable C═C function of the acrylate, methacrylate, maleate, fumarate, itaconate, citraconate, styrenic or vinylic type;
B) at least one polyol selected from oligoglycerols, linear, branched and hyperbranched polyglycerols or a mixture of hyperbranched polyglycerol and of a polyol selected from oligoglycerols and linear, branched and hyperbranched polyglycerols, comprising on average more than 1 hydroxyl group functionalized with a group bearing a polymerizable C═C function of the acrylate, methacrylate, maleate, fumarate, itaconate, citraconate, styrenic or vinylic type;
C) a radical initiator or a mixture of radical initiators capable of initiating polymerization by thermal, redox or photochemical routes; and
D) optionally at least one additive selected from antioxidants, agents for adjusting the physicochemical properties such as Young's modulus, elongation at break and breaking stress, agents for promoting, modulating and/or controlling permeability to oxygen, plasticizers, moistening agents, lubricants, viscosity lowering agents, compatibilizers, colorants, filtering agents, antimicrobial agents, therapeutic agents and agents against bacterial biofilms.

3. The composition as claimed in claim 1, in which the functionalized hyperbranched polyglycerol is obtained from a hyperbranched polyglycerol having a molecular weight between 546 and 100 000 g/mol, and has a degree of branching (DB) between 0.3 and 1.0.

4. The composition as claimed in claim 1, in which the hyperbranched polyglycerol is a polyglycerol dendrimer or a hyperbranched polyglycerol with a degree of branching between 0.3 and 1.0, or a mixture of hyperbranched polyglycerol and of oligoglycerols or of linear polyglycerols.

5. The composition as claimed in claim 1, in which the polymerizable C═C function on the functionalized hyperbranched polyglycerol is selected from the acrylic, methacrylic, maleic, fumaric, itaconic, citraconic or vinylic groups of structure: 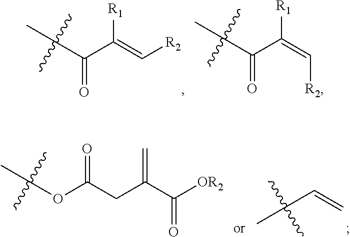
in which R₁ represents H or methyl; R₂ represents H, linear or branched C₁₋₆alkyl, or —C(═O)OR_{2A} in which R_{2A} represents H or linear or branched C₁₋₆alkyl optionally substituted with one or more hydroxyl groups, and R₃ represents H, linear or branched C1-20 alkyl.

6. The composition as claimed in claim 1, in constituent B), the polymerizable C═C function of the acrylate or methacrylate type is selected from acrylic acid, methacrylic acid, alkyl (meth)acrylates and derivatives thereof, such as 2-hydroxyethylmethacrylate (HEMA), 2-hydroxyethylacrylate (HEA), methylmethacrylate (MMA), methacrylamide, N,N-dimethyl-diacetoneacrylamide, 2-phosphatoethylmethacrylate, mono-, di-, tri-, tetra-, penta-polyethylene glycol acrylates or methacrylates, N-(3-methacrylamidopropyl)-N,N-dimethylamine, N-(3-methacrylamidopropyl)-N,N,N-trimethylamine, N-(3-acrylamido-3-methylbutyl)-N,N-dimethylamine, N-methacrylamide, 3-hydroxypropyl methacrylate, propyl methacrylate, propyl acrylate, butyl methacrylate, butyl acrylate, pentyl acrylate, pentyl methacrylate, N-(1,1-dimethyl-3-oxobutyl)acrylamide, 2-ethyl-2-(hydroxy-methyl)-1,3-propanediol trimethacrylate, butyl(meth)acrylate, 2-hydroxybutyl methacrylate, 3-hydroxy-2-naphthyl methacrylate, N-(formylmethyl)acrylamide, 2-ethoxyethyl methacrylate, 4-t-butyl-2-hydroxycyclohexyl methacrylate, the isocyanatoalkyl(meth)acrylates such as 2-isocyanatoethylmethacrylate, 2-isocyanatoethyl acrylate, and a compound selected from 4-[(a)-phenyl diazenyl]phenyl-2-methacrylate (4-[(E)-phenyldiazenyl]phenyl-2-methacrylate) and 4-[(a)-phenyl diazenyl]phenyl-2-methacrylate (4-[(E)-phenyldiazenyl]phenyl-2-methacrylate).

7. The composition as claimed in claim 6, in which the polymerizable C═C function is an isocyanatoalkyl(meth)acrylate.

8. The composition as claimed in claim 7, in which the isocyanatoalkyl(meth)acrylate is selected from 2-isocyanatoethylmethacrylate and 2-isocyanatoethylacrylate or a mixture thereof.

9. The composition as claimed in claim 1, in which constituent A) is selected from:
acrylic acid or methacrylic acid as well as alkyl (meth)acrylates and derivatives thereof, such as 2-hydroxyethylmethacrylate (HEMA), 2-hydroxyethylacrylate (HEA), methylmethacrylate (MMA), methacrylamide, N,N-dimethyl-diacetoneacrylamide, 2-phosphatoethylmethacrylate, mono-, di-, tri-, tetra-, penta-polyethylene glycol acrylates or methacrylates, N-(3-methacrylamidopropyl)-N,N-dimethylamine, N-(3-methacrylamidopropyl)-N,N,N-trimethylamine, N-(3-acrylamido-3-methylbutyl)-N,N-dimethylamine, N-methacrylamide, 3-hydroxypropyl methacrylate, propyl methacrylate, propyl acrylate, butyl methacrylate, butyl acrylate, pentyl acrylate, pentyl methacrylate, N-(1,1-dimethyl-3-oxobutyl)acrylamide, 2-ethyl-2-(hydroxy-methyl)-1,3-propanediol trimethacrylate, butyl(meth)acrylate, 2-hydroxybutyl methacrylate, 3-hydroxy-2-naphthyl methacrylate, N-(formylmethyl)acrylamide, 2-ethoxyethyl methacrylate, 4-t-butyl-2-hydroxycyclohexyl methacrylate, or the isocyanatoalkyl(meth)acrylates such as 2-isocyanatoethylmethacrylate, 2-isocyanatoethyl acrylate, 4-[(a)-phenyl diazenyl]phenyl-2-methacrylate (4-[(E)-phenyldiazenyl]phenyl-2-methacrylate) or 4-[(a)-phenyl diazenyl]phenyl-2-methacrylate (4-[(E)-phenyldiazenyl]phenyl-2-methacrylate),
the maleate and fumarate monomers, such as maleic acid, maleic anhydride, dimethyl maleate, diethyl maleate, or the higher maleate esters in which the alkyl chain contains 3 to 20 carbon atoms as well as fumaric acid, dimethyl fumarate, diethyl fumarate or the higher fumarate esters in which the alkyl chain contains 3 to 20 carbon atoms,
the itaconate monomers; such as itaconic acid, itaconic anhydride, dimethyl itaconate, or the higher itaconate esters in which the alkyl chain contains 3 to 20 carbon atoms,
the citraconate monomers; such as citraconic acid, citraconic anhydride, dimethyl citraconate, or the higher citraconate esters in which the alkyl chain contains 3 to 20 carbon atoms,
the (meth)acrylic monomers that may contain silicone or silane functions; for example tris(trimethylsiloxy)silylpropyl methacrylate, bis(trimethylsiloxy)methylsilylpropyl methacrylate, pentamethyldisiloxanepropyl methacrylate, tris(trimethylsiloxy) silylpropyloxyethyl methacrylate, tris(trimethylsiloxy)-silylpropyl methylacryloxyethylcarbamate, tris(trimethylsiloxy)silylpropylglycerol methacrylate, phenyltetramethyl-disiloxanylethyl acrylate, 3-methacryloxypropylbis(trimethylsiloxy)methylsilane, methyl-di(trimethylsiloxy)methacryloxymethyl silane, (3-methacryloxy-2-hydroxypropyloxy) propyl bis(trimethylsiloxy)methylsilane, 3-acryloxypropyl tris(trimethylsiloxy)silane, 3-acryloxypropyltrichlorosilane, 3-methacryloxypropyl dimethyl ethoxysilane, 3-methacryloxypropyl methyl diethoxysilane, methacryloxypropylbis(trimethylsiloxy)methylsilane, tristrimethylsilyloxysilylpropyl methacrylate, bis(methacryloxypropyl) tetramethyldisiloxane, acryloxymethyltrimethylsilane, p-(t-butyldimethylsiloxy)styrene, 1,3-bis(3-methacryloxypropyl)tetrakis(trimethylsiloxy)disiloxane, 3 trimethylsilylpropargylmethacrylate, 3-(trimethoxysilyl)propyl acrylate,
vinyl monomers, which may contain silicone or silane functions; for example vinyl(chloromethyl)dimethoxysilane, vinylethoxysiloxane-propylethoxysiloxane, vinyltris(1-methoxy-2-propoxy)silane, vinyltris(2-methoxyethoxy)silane, vinyltrimethoxysilane, vinyltriisopropoxysilane, vinyltriethoxysilane, vinyltrichlorosilane, vinyl tri-t-butoxysilane, o-(vinyloxybutyl)-n-triethoxysilylpropyl carbamate, vinylmethyldimethoxysilane, vinylmethyldiethoxysilane, vinyldimethylethoxysilane, 3-[tris(trimethylsiloxy)silyl] propylvinyl carbamate, triethoxysilylpropyl maleic acid or 1,3,5-trivinyl-1,1,3,5,5-pentamethyltrisiloxane,
fluorinated acrylic and methacrylic monomers, such as dihydroperfluorooctyl acrylate, 1,1-dihydroperfluorobutyl acrylate, trihydroperfluoroalkyl acrylate, tetrahydroperfluoroalkylacrylate, tris(trimethylsilyloxy)propyl methacrylate, perfluoro hexylethylthiocarbonylaminoethyl methacrylate, trifluoroethyl methacrylate, hexafluoroisopropyl methacrylate, hexafluorobutyl methacrylate, trifluoroethyl methacrylate,
organosilane prepolymer compounds, such as □□ω-bismethacryloxy-propyl polydimethylsiloxane, polydimethylsiloxanes with vinylic termination(s), diphenylsiloxane-dimethylsiloxane copolymers with a vinylic termination, trifluoropropylmethylsiloxane copolymers with vinylic termination(s), diethylsiloxane-dimethylsiloxane copolymers with vinylic termination(s), ethylsiloxane-dimethylsiloxane copolymers with vinylic termination(s), ethylene-siloxane copolymers with vinylic termination(s), tris(polydimethylsiloxy)silylpropyl methacrylate, polysiloxanylalkyl (meth)acrylates, methacryloxypropyl with polydimethylsiloxane termination, 1,3-divinyl-1,1,3,3-tetramethyldisiloxane, 2-(divinylmethylsilyl)ethyltriethoxysilane, or 1,3-divinyl-1,3-diphenyl-1,3-dimethyldisilane,
fluorinated/silane or chlorinated/silane mixed compounds, such as fluoro-methacryloxypropyl tris(trimethylsiloxy) silane, 3-acryloxy propyl methyl dichlorosilane, 3-acryloxypropyltrichlorosilane, 3-methacryloxypropyl trichlorosilane,
the silane/isocyanate compounds such as 3-isocyanatopropyltriethoxysilane, and
vinyl monomers, such as N-vinyl lactams; for example N-vinyl pyrrolidone (NVP)), N-vinyl-N-methyl acetamide (VMA), N-vinyl-N-ethyl acetamide, N-vinyl-N-ethyl formamide, N-vinyl formamide, N-2-hydroxyethyl vinyl carbamate, N-carboxy-alanine N-vinyl ester, preferably CH3C(O)N(CH3)-CH═CH2 (N-vinyl-N-methyl acetamide (VMA)).

10. The composition as claimed in claim 1, in which the initiator is at least one UV, thermal and/or redox initiator, selected from:
a photochemical initiator selected from the group comprising 2-hydroxy-4-acryloyloxyethoxy benzophenone, 2-hydroxy-4-(2-hydroxy-3-methacrylyloxy) propoxybenzophenone; benzophenone, acetophenone, acetonaphthoquinone, methyl ethyl ketone, valerophenone, hexanophenone, C-phenylbutyrophenone, p-morpholinopropiophenone, dibenzosuberone, 4-morpholinobenzophenone, 4-morpholinodeoxybenzoin, p-diacetylbenzene, 4-aminobenzophenone, para-methoxyacetophenone, 3-methylanthraquinone, tert-butylanthraquinone, the esters of anthraquinone, benzaldehyde, 9-acetylphenanthrene, 2-acetylphenanthrene, 10-thioxanthenone, 3-acetylphenanthrene, 3-acetylindole, 9-fluorenone, 1-indanone, 1,3,4-triacetylbenzene, thioxanthen-9-one, xanthen-9-one, 2,4-dimethylthioxanthone, 2,4-diethylthioxanthone, 2,4-diisopropylthioxanthone, 2,4-dichlorothioxanthone, benzoin, benzoin isobutyl ether, benzoin tetrahydropyranyl ether, benzoin methyl ether, benzoin ethyl ether, benzoin butyl ether, benzoin isopropyl ether, chloroxanthenone, 7-H-benzoin methyl ether, benzodeanthracen-7-one, 1-naphthaldehyde, 4,4′-bis(dimethylamino)benzophenone, 4-phenylbenzophenone, 4-chlorobenzophenone, 4,4′-bis(dimethylamino)benzophenone, 1-acetonaphthone, 2-acetonaphthone, 1-benzoylcyclohexan-1-ol, 2-hydroxy-2,2-dimethylacetophenone, 2′2-dimethoxy-2-phenylacetophenone, 2,2-diethoxy-2-phenylacetophenone, 1,1-dichloroacetophenone, 1-hydroxyacetophenone, acetophenone dimethyl acetal, o-methoxybenzophenone, triphenylphosphine, tris-o-tolylphosphine, benzaanthracene-7,12-dione, 2,2-diethoxyacetophenone, the benzyl acetals such as benzyl dimethyl acetal, 2-methyl-1-4-(methylthio)phenyl-2-morpholinopropan-1-one, the anthraquinones such as 2-methylanthraquinone, 2-ethylanthraquinone, 2-tert-butylanthraquinone, 1-chloroanthraquinone, 2-amylanthraquinone, 2,3-butanedione,
a photoinitiator selected from the group comprising esters of phenylglyoxalic acid, and combinations thereof with one another or with promoters of photopolymerization such as benzoic acid or amines,
a thermal initiator selected from the group comprising 2,2-azobis(2,4-dimethylpentanenitrile), 2,2′-azobisisobutyronitrile, 1,1′-azobis(cyclohexanecarbonitrile), 2,2′-azobis(2-methylpropionitrile), potassium peroxodisulfate, dibenzoyl peroxide, cyclohexanone peroxide, di-tert-butyl peroxide, acetyl cyclohexylsulfonyl peroxide, diisopropyl percarbonate, tert-butyl peroctoate or benzpinacol, di-t-butyl peroxide, cumene hydroperoxide, dicumyl peroxide, t-butyl perbenzoate, and hydroxylated N-oxide amine, such as 2,2,6,6-tetramethylpiperidine-N-oxyl and 4-hydroxy-2,2,6,6-tetramethylpiperidine-N-oxyl,
a redox initiator system selected from the group comprising oxidants such as AIBN, the peroxides such as hydrogen peroxide, benzoyl peroxide, t-butyl-hydroperoxide, p-mentane hydroperoxide, ammonium persulfate, sodium persulfate, potassium persulfate, reducing agents such as ascorbic acid, sodium bisulfite, N,N,N′,N′-tetramethylethylene diamine formaldehyde sodium sulfoxylate, the Fe2+, Cr2+, Zn2+, V2+, Ti3+, CO2+, Cu+ metal salts such as iron sulfate, and the N,N-dialkylanilines such as N,N-dimethyl-p-toluidine.

11. A hydrogel obtainable by:
hydration/swelling, from water or an aqueous solution, of a crosslinked composition as claimed in claim 2, or,
polymerization of the crosslinkable composition as claimed in claim 1, in the presence of a water-miscible protic or aprotic solvent, and then exchange of the solvent with an excess of water or an aqueous solution, or
by polymerization of the crosslinkable composition as claimed in claim 1, in the presence of water or an aqueous solution.

12. A method for preparing a crosslinked composition as defined in claim 2 consisting of reacting, in the presence or absence of aprotic solvent, preferably in the absence of solvent:
A) at least one polymerizable monomer or mixture of polymerizable monomers or a mixture of monomers and polymerizable macromonomers bearing at least one polymerizable C═C function of the acrylate, methacrylate, maleate, fumarate, itaconate, citraconate, styrenic or vinylic type
B) at least one polyol selected from oligoglycerols and linear, branched and hyperbranched polyglycerols, or a mixture of hyperbranched polyglycerol and of a polyol selected from oligoglycerols and linear, branched and hyperbranched polyglycerols, comprising on average more than 1 hydroxyl groups functionalized with a group bearing a polymerizable C═C function of the acrylate, methacrylate, maleate, fumarate, itaconate, citraconate, styrenic or vinylic type;
C) a radical initiator or a mixture of radical initiators that are able to initiate polymerization by thermal, redox or photochemical routes; and
D) optionally at least one additive selected from antioxidants, agents for adjusting the physicochemical properties such as Young's modulus, elongation at break and breaking stress, promoters of permeability to oxygen, plasticizers, moistening agents, lubricants, viscosity modifiers, compatibilizers, colorants, filtering agents, antimicrobial agents, therapeutic agents and agents against bacterial biofilms.

13. The method as claimed in claim 12, in which the functionalized hyperbranched polyglycerol is present at a rate from 0.1 to 99.99% by weight of the total weight of constituents A) to D), or in which the initiator is present at a rate from 0.02 to 5% by weight of the total weight of constituents A) to D).

14. The method as claimed in claim 12, further comprising a step of molding the crosslinked composition.

15. An article obtainable by the method as claimed in claim 12.

16. The article as claimed in claim 15, which is selected from a contact lens or intraocular lens, a patch, an implant or a dressing.

17. The use of a composition or of a hydrogel as claimed in claim 1 for manufacturing a medical device, or as a vector of a compound of interest selected from therapeutic active ingredients, vitamins, nutrients, decontaminating agents or lubricants, or in biomedical, cosmetic, or health-related applications.

18. The use of a hyperbranched polyglycerol comprising on average more than one group, preferably at least 2 hydroxyl groups functionalized with a group bearing a polymerizable C═C function, as crosslinking agent, comonomer or principal monomer for manufacturing a contact lens or intraocular lens.
